(19) United States
(12) Patent Application Publication
(10) Pub. No.: US 2014/0155324 A1
(43) Pub. Date: Jun. 5, 2014

(54) NOVEL PEPTIDES AND USES THEREOF IN THERAPEUTIC METHODS

(71) Applicants: Tamara Sandler, Jerusalem (IL); Orly Devary, Jerusalem (IL)

(72) Inventors: Tamara Sandler, Jerusalem (IL); Orly Devary, Jerusalem (IL)

(73) Assignee: TWO TO BIOTECH LTD., Jerusalem (IL)

(21) Appl. No.: 14/235,903

(22) PCT Filed: Aug. 6, 2012
(86) PCT No.: PCT/IL2012/000295

§ 371 Date: Jan. 29, 2014

Related U.S. Application Data
(60) Provisional application No. 61/515,991, filed on Aug. 8, 2011.

(51) Int. Cl. C07K 14/47

(52) U.S. Cl. CPC C07K 14/47
USPC 514/7.3; 435/325; 435/320.1; 514/6.8; 514/6.9; 530/324; 530/327; 536/23.1

(57) ABSTRACT

The invention relates to polypeptides exhibiting metabolic-modulating as well as immunomodulatory properties, nucleic acids encoding them, compositions comprising them, and methods of treating metabolic disorders and/or immune-related disorders comprising their administration and to their use in preparing pharmaceutical compositions for the treatment, amelioration, prophylaxis or delaying the onset of at least one of a metabolic disorder and an immune-related disorder.

### FIELD OF THE INVENTION

The invention relates to novel peptides exhibiting metabolic-modulating as well as immunomodulatory properties. More specifically, the invention provides novel peptides capable of ameliorating metabolic parameters relevant to diabetes and its pathogenesis, as well as other metabolic disorders such as the metabolic syndrome, as well as to immune-related disorders and cardiac disorders. The invention is also directed to compositions comprising the peptides, to methods of treatment comprising their administration and to their uses in the preparation of said compositions.

### BACKGROUND OF THE INVENTION

The discussion in this section is not limited to subject matter that qualifies as “prior art” against the present invention. Therefore, no admission of such prior art status shall be implied or inferred by reason of inclusion of particular subject matter in this discussion, and no declaration against the present inventors' interests shall be implied by reason of such inclusion.

All publications mentioned throughout this application are fully incorporated herein by reference, including all references cited therein.

Disorders of carbohydrate metabolism occur in many forms. The most common disorders are acquired. Acquired or secondary derangements in carbohydrate metabolism, such as diabetic ketoacidosis, hyperosmolar coma, and hypoglycemia, all affect the central nervous system. Many forms and variants of peripheral nerve disease also are seen in diabetes. The remaining disorders of carbohydrate metabolism are the rare inborn errors of metabolism (i.e. genetic defects).

The acquired disorders of carbohydrate metabolism are fairly common, both in the United States and internationally. Hypoglycemia is a common cause of neurological disease, especially acute mental deterioration, memory loss, disorientation, obtundation, and coma, among both alcoholics and patients with diabetes who are treated with insulin. Hyperinsulinemia from other causes is rare, but pancreatic tumors could be the cause.

Diabetes (diabetes mellitus) is the most common endocrine disease, and is characterized by abnormalities of glucose metabolism. The abnormal glucose metabolism associated with this disease results in hyperglycemia (high blood glucose levels) and eventually causes complications of multiple organ systems, including eyes, kidneys, nerves, and blood vessels. Patients with persistent hyperglycemia or abnormal glucose tolerance are generally diagnosed with the disease, although most commonly patients initially present with excessive urination (polyuria) and frequent drinking due to extreme thirst (polydipsia). These typical initial symptoms result from the osmotic effects of hyperglycemia.

The pathogenesis of diabetes mellitus is typically associated with pancreatic dysfunction, particularly of the beta cells of the pancreatic islets of Langerhans. This dysfunction may lead to destruction of the islet beta cells, which produce insulin, a glucose regulatory peptide hormone. Diabetes mellitus has been generally categorized as insulin dependent or type I, versus non-insulin dependent, or type II.

The principal three forms or diabetes are:
Type I: Results from the body's failure to produce insulin. Treatment usually involves insulin administration.
Type II: Results from a condition in which the body fails to use insulin properly, combined with relative insulin deficiency. Many people destined to develop type II diabetes spend many years in a state of Pre-diabetes, a condition that occurs when a person's blood glucose levels are higher than normal but not high enough for a diagnosis of type II diabetes.
Gestational diabetes: Pregnant women who have never had diabetes before but who have high blood sugar (glucose) levels during pregnancy are said to have gestational diabetes. Gestational diabetes affects about 4% of all pregnant women. It may precede development of type II (or rarely type I).
Many other forms of diabetes are categorized separately from these. Examples include congenital diabetes due to genetic defects of insulin secretion, cystic fibrosis-related diabetes, steroid diabetes induced by high doses of glucocorticoids, and several forms of monogenic diabetes.

However, this terminology has evolved as the disease has become better understood. For example, it has been found that in some patients suffering from non-insulin dependent diabetes, the disease progresses into an insulin dependent form, while in other patients insulin dependence does not develop.

Patients are thus often categorized in terms of the mechanisms of pathogenesis of islet destruction, and the designation type I is now used to refer to autoimmune islet pathogenesis, i.e., to diabetes caused by islet-specific autoimmune attack, and is so used herein. The term insulin dependent diabetes mellitus (IDDM) refers to Type I diabetes that has progressed to a stage where enough autoimmune destruction of the pancreatic beta cells has occurred to produce overt symptoms. The term pre-IDDM refers to an autoimmune condition that can be detected by biopsy or by analysis of autoimmune responses, in which pancreatic islet beta cells are being subject to a specific autoimmune attack to an extent where some cells may be subject to destruction. In pre-IDDM, however, the destruction (if any) has not progressed to an extent sufficient to require the administration of insulin. Since there can be a point in the early stages of Type I diabetes in which overt symptoms are observed but some islet function remains (known as the “honeymoon period”), not all Type I diabetes is classified as IDDM, and not all pre-IDDM presents without overt symptoms.

The metabolic complications associated with the abnormal metabolism caused by insulin insufficiency can affect numerous organ systems. The most common acute metabolic complication is that of diabetic ketoacidosis, characterized by severe hyperglycemia (and resulting hypovolemia caused by osmotic diuresis) as well as metabolic acidosis induced by excess free fatty acid release and the production of ketone bodies.

In addition to the acute metabolic complication of ketoacidosis, the diabetic patient is susceptible to a series of late complications that cause considerable morbidity and premature mortality. Atherosclerosis occurs more extensively and earlier in diabetics than in the general population as a result of abnormalities in both glucose and lipid metabolism. This vascular pathology can lead to, inter alfa, coronary artery disease, stroke, and peripheral vascular disease with gangrene. Retinopathy is another vascular complication of diabetes. Diabetic retinopathy is a leading cause of blindness, and is initiated by increased permeability of retinal capillaries which can progress to occlusion, hemorrhage, aneurysm formation, and neovascularization known as proliferative retinopathy.

As mentioned above, meticulous control of blood glucose has been associated with amelioration of the late complications of Type I diabetes, suggesting that preservation or restoration of beta cell function could reduce or eliminate the majority of the pathologic complications of the disease.

The inherited disorders of carbohydrate metabolism are rare. Severe defects of the pyruvate dehydrogenase (PDH) complex and the benign chemical anomaly called pentosuria have been reported in very few patients.

Hypoglycemia, diabetic ketoacidosis, and hyperosmolar coma are all potentially fatal but potentially curable conditions.

Disorders of carbohydrate metabolism are commonly associated with other metabolic disorders, forming together the metabolic syndrome. Metabolic syndrome is a combination of medical disorders that, when occurs together, increase the risk of developing cardiovascular disease and diabetes. It affects one in five people in the United States and prevalence increases with age. Some studies have shown the prevalence in the USA to be an estimated 25% of the population.

In the present application, the inventors present novel peptides demonstrating a surprising capacity to improve metabolic indices, thus ameliorating the condition of patients suffering from a metabolic disorder, preventing deterioration in said patients, or delaying the onset or providing effective prophylaxis for such disorders.

Accordingly, it is an object of the invention to provide the peptides of the invention. The present invention also provides compositions comprising said peptides, as well as their uses in therapeutics.

These and other uses and objects of the invention will become apparent as the description proceeds.

### SUMMARY OF THE INVENTION

In the first aspect, the invention provides an isolated polypeptide comprising an amino acid sequence denoted by any one of SEQ. ID. NO. 1, SEQ. ID. NO. 5 or SEQ. ID. NO. 9, and any fragments, derivatives or analogues thereof. It should be noted that said polypeptides are hormone-like proteins, and are also referred to herein as APC2, PRT10 or UZI#1; APC3, PRT9 or UZI#2; and APC4, PRT11 or UZI#3, respectively.

In the second aspect, the invention provides an isolated nucleic acid molecule comprising a sequence encoding a polypeptide comprising an amino acid sequence denoted by any one of SEQ. ID. NO. 1, SEQ. ID. NO. 5 or SEQ. ID. NO. 9, and any fragments, derivatives and analogs thereof. It should be noted that non-limiting examples of fragments of the peptides of the invention are also denoted by SEQ ID NO. 13, 14 and 15, respectively.

The invention further provides nucleic acid constructs comprising a nucleic acid sequence denoted by any one of SEQ. ID. NO. 2, SEQ. ID. NO. 6 or SEQ. ID. NO. 10, or any derivative, mutant, fragment or homolog thereof. The construct optionally further comprises operably linked regulatory elements. Still further, the invention provides expression vectors comprising the nucleic acid construct according to the invention, as well as host cell transformed or transfected with the expression vector according to the invention.

In yet another aspect, the invention provides a composition comprising at least one isolated polypeptide comprising an amino acid sequence denoted by any one of SEQ. ID. NO. 1, SEQ. ID. NO. 5 or SEQ. ID. NO. 9, and any fragments, derivatives or analogs thereof, or a nucleic acid sequence encoding the same. The composition optionally further comprises a pharmaceutically acceptable carrier, diluent or excipient.

Furthermore, according to certain embodiments, the invention provides a pharmaceutical composition for the treatment, amelioration, prophylaxis or delaying the onset of at least one of a metabolic disorder, cardiac disorders and an immune-related disorder. The pharmaceutical compositions of the invention comprise at least one isolated polypeptide comprising an amino acid sequence denoted by any one of SEQ. ID. NO. 1, SEQ. ID. NO. 5 or SEQ. ID. NO. 9, and any fragments, derivatives or analogs thereof, or a nucleic acid sequence encoding the same, and a pharmaceutically acceptable carrier, diluent or excipient.

In a further aspect, the invention provides a method for the treatment, amelioration, prophylaxis or delaying the onset of at least one of a metabolic disorder, cardiac disorders and an immune-related disorder. The method comprises the step of administering to a subject in need thereof a therapeutically effective amount of at least one isolated polypeptide comprising an amino acid sequence denoted by any one of SEQ. ID. NO. 1, SEQ. ID. NO. 5 or SEQ. ID. NO. 9, or any fragments, derivatives or analogs thereof, a nucleic acid sequence encoding the same, any combinations or mixtures thereof or any composition comprising the same.

In a further aspect, the invention is directed to the use of at least one isolated polypeptide comprising an amino acid sequence denoted by any one of SEQ. ID. NO. 1, SEQ. ID. NO. 5 or SEQ. ID. NO. 9, and any fragments, derivatives or analogs thereof, or a nucleic acid sequence encoding the same, in the preparation of a composition. The composition is suitable for the treatment, amelioration, prophylaxis or delaying the onset of at least one of a metabolic disorder, cardiac disorders and an immune-related disorder.

In yet another aspect, the invention provides an isolated polypeptide for use in the treatment, amelioration, prophylaxis or delaying the onset of at least one of an immune-related disorder, cardiac disorders and a metabolic disorder. The polypeptide comprises an amino acid sequence denoted by any one of SEQ. ID. NO. 1, SEQ. ID. NO. 5 or SEQ. ID. NO. 9, and any fragments, derivatives or analogs thereof.

The invention also provides a method for enhancing glucose metabolism, increasing glucose tolerance, reducing plasma glucagon levels, inducing insulin receptor expression, increasing GLUT-4 glucose transporter expression, reducing serum levels of TNFα, and reducing serum levels of IL-1β, the method comprises the step of administering to a subject in need thereof a therapeutically effective amount of at least one isolated polypeptide comprising an amino acid sequence denoted by any one of SEQ. ID. NO. 1, SEQ. ID. NO. 5 or SEQ. ID. NO. 9, or any fragments, derivatives or analogs thereof, a nucleic acid sequence encoding the same, any combinations or mixtures thereof or any composition comprising the same.

These and other aspects of the invention will become apparent by the hand of the proceeding drawings.

### BRIEF DESCRIPTION OF THE FIGURES

FIG. 1

The APC-2 peptide effects on glucose tolerance

Mouse blood glucose levels in mice treated as described with vehicle (pink), 0.25 mg/Kg (dark blue line), 1.5 mg/Kg (yellow line), or 15 mg/Kg (light blue line) APC-2, after administration of intraperitoneal (2 g/Kg body weight) glucose injection are shown.

SD of all points was lower than 12%.

FIG. 2

The APC-2peptide effects on plasma glucagon levels

Mouse blood glucagon levels in mice treated as described with indicated APC-2 dosages are shown.

SD of all points was lower than 12%.

FIG. 3A-3C

The APC-2 peptide increases Insulin Receptor and GLUT-4 expression in splenocytes

Northern blot analysis of HPRT, insulin receptor and GLUT-4 in mice treated as described with indicated APC-2 dosages (C [control]; 5 [0.25 mg/Kg body weight]; 30 [1.5 mg/Kg body weight]; 300 [15 mg/Kg body weight]) are shown.

FIG. 3A: HPRT Northern blot analysis.

FIG. 3B: Insulin receptor Northern blot analysis.

FIG. 3C: GLUT-4 Northern blot analysis.

FIG. 4A-4B

The APC-3 peptide reduces levels of TNFα and IL-1β

Serum cytokine levels in mice treated as described with indicated APC-3 dosages are shown.

FIG. 4A: TNFα serum levels analysis.

FIG. 4B: IL-1β serum levels analysis.

### DETAILED DESCRIPTION OF THE INVENTION

The invention herein provides novel peptides performing modulatory properties of metabolic and immune-related parameters. Thus, in the first aspect, the invention provides an isolated polypeptide comprising an amino acid sequence denoted by any one of SEQ. ID. NO. 1, SEQ. ID. NO. 5 or SEQ. ID. NO. 9, and any fragments, derivatives or analogues thereof.

The term “peptide”, which also may be referred to as “protein” or “polypeptide” interchangeably herein, refers to a polymer of amino acid residues and is not limited to a minimum length of the product. Thus, peptides, oligopeptides, dimers, multimers, and the like, are included within the definition. Both full-length proteins and fragments thereof are encompassed by the definition. The terms also include post translation expression modifications of the polypeptide, for example, glycosylation, acetylation, phosphorylation and the like. Furthermore, for purposes of the present invention, a “peptide” or “protein” refers to a peptide which includes modifications, such as deletions, additions and substitutions (generally conservative in nature), to the native sequence, so as long as the protein maintains the desired activity, i.e., induction of immune and/or metabolic effects as described in the specification, namely, increasing glucose tolerance, reducing plasma glucagon levels, increasing insulin receptor expression, increasing GLUT-4 glucose transporter expression and reducing serum levels of TNFα and reducing serum IL-1β. These modifications may be deliberate, as through site-directed mutagenesis, or may be accidental, such as through mutations of hosts which produce the proteins or errors due to PCR amplification of nucleic acids coding for said proteins.

The term “isolated” or “purified” refers to molecules, such as amino acid sequences, or peptides that are removed from their natural environment, isolated or separated. An “isolated peptide” is therefore a purified amino acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample.

The term “amino acids” as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Amino acid sequence” or “peptide sequence” is the order in which amino acid residues, connected by peptide bonds, lie in the chain in peptides and proteins. The sequence is generally reported from the N-terminal end containing free amino group to the C-terminal end containing free carboxyl group. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, sulfhydryl bond formation, cleavage and the likes.

It should be appreciated that the invention provides specific peptides but also encompasses any functional fragment of these peptides. A “fragment” constitutes a fraction of the amino acid or DNA sequence of a particular region. A fragment of the peptide sequence is at least one amino acid shorter than the particular region, and a fragment of a DNA sequence is at least one base-pair shorter than the particular region. The fragment may be truncated at the C-terminal or N-terminal sides, or both. An amino acid fragment may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 24, at least 26, at least 27 or at least 28 amino acid residues of SEQ ID NO. 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 24, at least 26, at least 27 or at least 28, at least 29, at least 30, at least 31, at least 32, at least 33 or at least 34, at least 35, at least 36, at least 37, at least 38, at least 39, at least 40 amino acids of SEQ ID NO. 5, and at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 24, at least 26, at least 27 or at least 28, at least 29, at least 30, at least 31, at least 32, at least 33 or at least 34, at least 35, at least 36, at least 37, at least 38, at least 39, at least 40, at least 41, at least 42, at least 43, at least 44, at least 45, or at least 46 amino acids of SEQ ID NO. 9.

Non limiting examples of functional fragments may include the peptides of SEQ ID NO. 13, 14 and 15, that are functional fragments of the peptides of SEQ ID NO. 1, 5, and 9, respectively. More specifically, the peptide of SEQ ID NO. 13 is a functional fragment of the APC2 peptide as denoted by SEQ ID NO. 1. In another embodiment, the peptide of SEQ ID NO. 14 is a functional fragment of the APC3 peptide as denoted by SEQ ID NO. 5. Still further, the peptide of SEQ ID NO. 15 is a functional fragment of the APC4 peptide as denoted by SEQ ID NO. 9.

An “analog” of a molecule can be a homologous molecule from the same species or from different species. The amino acid sequence of an analogue or derivative may differ from the original sequence, when at least one residue is deleted, inserted or substituted.

Still further, the invention concerns derivatives of the amino acid sequence of the invention. Derivatives of the amino acid sequences of the invention are, for example, where functional groups, such as amino, hydroxyl, mercapto or carboxyl groups, are derivatised, e.g. glycosylated, acylated, amidated or esterified, respectively. In glycosylated derivatives an oligosaccharide is usually linked to asparagine, serine, threonine and/or lysine. Acylated derivatives are especially acylated by a naturally occurring organic or inorganic acid, e.g. acetic acid, phosphoric acid or sulphuric acid, which usually takes place at the N-terminal amino group, or at hydroxy groups, especially of tyrosine or serine, respectively. Esters are those of naturally occurring alcohols, e.g. methanol or ethanol. Further derivatives are salts, especially pharmaceutically acceptable salts, for example metal salts, such as alkali metal and alkaline earth metal salts, e.g. sodium, potassium, magnesium, calcium or zinc salts, or ammonium salts formed with ammonia or a suitable organic amine, such as a lower alkylamine, e.g. triethylamine, hydroxy-lower alkylamine, e.g. 2-hydroxyethylamine, and the like.

It should be appreciated that by the term “insertions”, as used herein it is meant any addition of amino acid residues to the sequence of the invention, of between 1 to 50 amino acid residues, specifically, between 20 to 1 amino acid residues, and more specifically, between 1 to 10 amino acid residues. Most specifically, 1, 2, 3, 4, 5, 6, 7, 8, 9 and 10 amino acid residues. Further, the amino acid sequence of the invention may be extended at the N-terminus and/or C-terminus thereof with various identical or different amino acid residues.

Amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

With respect to amino acid sequences, one of skill will recognize that individual substitutions, deletions or additions to an amino acid, nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M).

Conservative nucleic acid substitutions are nucleic acid substitutions resulting in conservative amino acid substitutions as defined above.

Variants of the amino acid sequences of the invention may have at least 80% sequence similarity, often at least 85% sequence similarity, 90% sequence similarity, or at least 95%, 96%, 97%, 98%, or 99% sequence similarity at the amino acid level, with any one of the peptides denoted as SEQ ID NO.: 1, 5 and 9, respectively.

It is understood that the invention relates to any functional fragments, derivatives or analogues of the amino acid sequences denoted as SEQ ID NO.: 1, 5 and 9. As used herein, the terms “functional fragment”, “functional derivatives” or “functional analogues” refers to an amino acid sequence which possesses biological function or activity that is identified through a defined functional assay. More specifically, the defined functional assay is an assay for any of the parameters affected by the peptides of the invention, namely, glucose tolerance, plasma glucagon levels, insulin receptor expression, GLUT-4 glucose transporter expression and serum levels of TNFα and IL-1β.

According to one embodiment, the isolated polypeptide of the invention comprises an amino acid sequence denoted by SEQ. ID. NO. 1, or any fragment, derivative or analogue thereof. Said polypeptide is a hormone-like protein, and is also referred to herein as APC2, PRT10 or UZI#1.

In certain embodiments, the peptide of the invention is expressed in at least one of human leukocytes and heart.

“Expression” is interpreted here as the process of transcription and/or translation of a gene to mRNA and to polypeptide. More particularly, when referring to the expression of a peptide in a specific tissue or tissues, what is meant that said transcript and/or peptide is expressed in a significant quantity which is functional, i.e., induces relevant effects.

According to other embodiments, the isolated polypeptide of the invention comprises an amino acid sequence denoted by SEQ. ID. NO. 5, or any fragment, derivative or analogue thereof. Said polypeptide is a hormone-like protein, and is also referred to herein as APC3, PRT9 or UZI#2.

In certain embodiments, the peptide of the invention is expressed in at least one of human spleen, testis, small intestine, colon and kidney.

According to further embodiments, the isolated polypeptide of the invention comprises an amino acid sequence denoted by SEQ. ID. NO. 9, or any fragment, derivative or analogue thereof. Said polypeptide is a hormone-like protein, and is also referred to herein as APC4, PRT11 or UZI#3.

In certain embodiments, the peptide of the invention is expressed in at least one of human spleen, testis, colon, small intestine, leukocytes, heart, placenta, liver, kidney and pancreas

In certain embodiments, the peptides of the invention may be hormone-like secreted peptides.

As demonstrated by the following examples, the peptides of the invention are capable of at least one of: increasing glucose tolerance, reducing plasma glucagon levels, increasing insulin receptor expression, increasing GLUT-4 glucose transporter expression and reducing serum levels of TNFα and reducing serum IL-1β.

Generally, when used, the terms increase, elevate or augment relate to the induction of an increase, elevation or augmentation in a value, a process, a phenomenon or a phenotype referred to, such as for example, serum levels of certain compounds. Said increase, elevation or augmentation may also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 100%, or even more.

The term “inhibit” or “inhibition”, as used herein, means the restriction, retardation, reduction, decrease or diminishing of a process, a phenomenon or a phenotype by at least about 1%400%, about 5%-95%, about 10%-90%, about 15%-85%, about 20%-80%, about 25%-75%, about 30%-70%, about 35%-65%, about 40%-60% or about 45%-55%. Said restriction, retardation, reduction, decrease or diminishing of a process, a phenomenon or a phenotype may also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

Therefore, in certain embodiments, the peptides of the invention may be effective for the treatment, amelioration, prophylaxis or delaying the onset of an immune-related disorder or a metabolic disorder.

In specific embodiments, the peptide of the invention are effective for the treatment, amelioration, prophylaxis or delaying the onset of the metabolic syndrome or of any of the conditions comprising the same. More specifically Metabolic Syndrome or any of the conditions comprising the same may be at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

In other embodiments, the peptides of the invention are capable of modulating the Th1/Th2, Th3 cell balance in a subject in need thereof.

In some embodiments, the peptides of the invention are immunomodulatory. The term “modulate” or “modulating” as used herein, for instance such as modulating an immune response or condition, encompasses the increase or decrease of activity or response in relation to a control or the normal or baseline level of activity or response under certain conditions. It can also encompass the maintaining of a level of activity or response under conditions that would normally increase or decrease the level of activity of the peptide or response.

In certain specific embodiments, the peptides of the invention may modulate the Th1/Th2, Th3 cell balance towards an anti-inflammatory Th2, Th1/Th3 response may be particularly applicable in immune related disorders having an undesired unbalanced pro-inflammatory Th1 reaction. For example, such immune-related disorders may be an autoimmune disease, graft rejection pathology and an inflammatory disease.

The present invention provides novel peptides having metabolic modulatory properties. In should be therefore appreciated that furthermore, the invention also relates to the nucleic acid sequences provided by the invention, and those encoding the above amino acid sequences.

Thus, in the second aspect, the invention provides an isolated nucleic acid molecule comprising a sequence encoding a polypeptide comprising an amino acid sequence denoted by any one of SEQ. ID. NO. 1, SEQ. ID. NO. 5 or SEQ. ID. NO. 9, and any fragments, derivatives and analogs thereof.

As used herein, the term “nucleic acid” refers to polymer of nucleotides, which may be either single- or double-stranded, which is a polynucleotide such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides. The term DNA used herein also encompasses cDNA, i.e. complementary or copy DNA produced from an RNA template by the action of reverse transcriptase (RNA-dependent DNA polymerase).

According to some embodiments, the isolated nucleic acid molecule of the invention, which comprises the nucleic acid sequence denoted by SEQ ID. NO. 2 or any derivative, mutant, fragment or homolog thereof, encodes a polypeptide comprising an amino acid sequence denoted by SEQ. ID. NO. 1. Said polypeptide is a hormone-like protein, and is also referred to herein as APC2, PRT10 or UZI#1.

According to other embodiments, the isolated nucleic acid molecule of the invention, which comprises the nucleic acid sequence denoted by SEQ ID. NO. 6 or any derivative, mutant, fragment or homolog thereof, encodes a polypeptide comprising an amino acid sequence denoted by SEQ. ID. NO. 5. Said polypeptide is a hormone-like protein, and is also referred to herein as APC3, PRT9 or UZI#2.

According to further embodiments, the isolated nucleic acid molecule of the invention, which comprises the nucleic acid sequence denoted by SEQ ID. NO. 9 or any derivative, mutant, fragment or homolog thereof, encodes a polypeptide comprising an amino acid sequence denoted by SEQ. ID. NO. 10. Said polypeptide is a hormone-like protein, and is also referred to herein as APC4, PRT11 or UZI#3.

According to certain embodiments the invention further provides isolated nucleic acid molecules that only differ from the nucleic acid molecules denoted by any one of SEQ ID NO. 2, 6 and 10 in codon sequence due to the degeneracy of the genetic code.

Due to the degenerative nature of the genetic code it is clear that a plurality of different nucleic acid sequences can be used to code for the amino acid sequences of the invention. It should be appreciated that the codons comprised in the nucleic acid sequence of the invention may be optimized (codon-optimized) for expression in any specific host cell, preferably in host cells capable of expressing large quantities of the desired peptides, and most preferably, commercial quantities of said peptides.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

It should be appreciated that the invention further encompasses any derivative, mutant, fragment or homolog of the nucleic acid sequences provided by the invention. Specifically, an analogue or derivative of the nucleic acid sequence that may be used by the methods, compositions and uses of the invention may comprise at least one mutation, point mutation, nonsense mutation, missense mutation, deletion, insertion or rearrangement.

A nucleic acid fragment of a sequence according to the invention may comprise at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 101, at least 102, at least 103, at least 104 or at least 121 nucleic acids, or even more.

In the third aspect, the invention provides a nucleic acid construct comprising a nucleic acid sequence denoted by any one of SEQ. ID. NO. 2, SEQ. ID. NO. 6 or SEQ. ID. NO. 10, or any derivative, mutant, fragment or homolog thereof. The construct optionally further comprises operably linked regulatory elements.

“Construct”, as used herein, encompasses vectors such as plasmids, viruses, bacteriophage, integratable DNA fragments, and other vehicles, which enable the integration of DNA fragments into the genome of the host.

Still further, the invention also provides an expression vector comprising the nucleic acid construct according to the invention.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

In a further aspect, the invention is directed to a host cell transformed or transfected with the expression vector according to the invention.

“Cells”, “host cells” or “recombinant host cells” are terms used interchangeably herein. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein.

“Host cell” as used herein refers to cells which can be recombinantly transformed with naked DNA or expression vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

The host cells of the invention are transformed or transfected with the expression vector descried herein to express the peptides of the invention. “Transformation”, as used herein, refers to a process in which a cell's genotype is changed as a result of the cellular uptake of exogenous DNA or RNA, and, for example, the transformed cell expresses a recombinant form of at least one of the peptides of the invention. The term “transfection” means the introduction of a nucleic acid, e.g., naked DNA or an expression vector, into a recipient cells by nucleic acid-mediated gene transfer.

In yet another aspect, the invention provides a composition comprising at least one isolated polypeptide comprising an amino acid sequence denoted by any one of SEQ. ID. NO. 1, SEQ. ID. NO. 5 or SEQ. ID. NO. 9, and any fragments, derivatives or analogs thereof, or a nucleic acid sequence encoding the same. The composition optionally further comprises a pharmaceutically acceptable carrier, diluent or excipient.

In various embodiments, the polypeptide comprised in the composition of the invention comprises an amino acid sequence denoted by SEQ. ID. NO. 1, or any fragments, derivatives or analogs thereof. Said polypeptide is a hormone-like protein, and is also referred to herein as APC2, PRT10 or UZI#1.

In a number of embodiments, the polypeptide comprised in the composition of the invention comprises an amino acid sequence denoted by SEQ. ID. NO. 5, or any fragments, derivatives or analogs thereof. Said polypeptide is a hormone-like protein, and is also referred to herein as APC3, PRT9 or UZI#2.

In further embodiments, the polypeptide comprised in the composition of the invention comprises an amino acid sequence denoted by SEQ. ID. NO. 9, or any fragments, derivatives or analogs thereof. Said polypeptide is a hormone-like protein, and is also referred to herein as APC4, PRT11 or UZI#3.

As demonstrated in the Examples, the peptides of the invention, and therefore also the composition comprising the same, may lead to at least one of: enhancing glucose metabolism, increasing glucose tolerance, reducing plasma glucagon levels, inducing insulin receptor expression, increasing GLUT-4 glucose transporter expression, reducing serum levels of TNFα and reducing serum levels of IL-1β. As can be appreciated, the peptides and composition of the invention impart an anti-inflammatory and metabolically-favorable effect. This effect may be particularly suitable for the treatment or prophylaxis of immune-related metabolic disease, such as diabetes and complications associated with the metabolic syndrome.

To take advantage of the beneficial effects of the peptides and compositions of the invention, a composition suitable for administration is required. Therefore, in some embodiments, the composition of the invention is a pharmaceutical composition for the treatment, amelioration, prophylaxis or delaying the onset of at least one of a metabolic disorder, a cardiac disorders and an immune-related disorder.

According to certain embodiments, the pharmaceutical composition of the invention is for the treatment, amelioration, prophylaxis or delaying the onset of the metabolic syndrome or of any of the conditions comprising the same.

In certain embodiments, it should be noted that Metabolic Syndrome or any of the conditions comprising the same may include at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

More specifically, as indicated herein an example for a “metabolic disorder” is the Metabolic Syndrome. The Metabolic Syndrome is characterized by a group of metabolic risk factors in one person including:

Abdominal obesity (excessive fat tissue in and around the abdomen); Atherogenic dyslipidemia (blood fat disorders—high triglycerides, low HDL cholesterol and high LDL cholesterol—that foster plaque buildups in artery walls); elevated blood pressure; insulin resistance or glucose intolerance; prothrombotic state (e.g., high fibrinogen or plasminogen activator inhibitor-1 in the blood); and pro-inflammatory state (e.g., elevated C-reactive protein in the blood). People with the metabolic syndrome are at increased risk of coronary heart disease and other diseases related to plaque buildups in artery walls (e.g., stroke and peripheral vascular disease) and type 2 diabetes.

As indicated above, metabolic syndrome is a combination of medical disorders that, when occurring together, increase the risk of developing cardiovascular disease and diabetes. Some studies have shown the prevalence in the USA to be an estimated 25% of the population. As indicate herein before, there are many different medical criteria for the syndrome, but in general, it may include one or more of the following abnormal medical parameters: increased central obesity, dyslipidemia (as manifested, for example in high triglyceride levels and/or low HDL-C levels), hypertension, high fasting plasma glucose, microalbuminuria, and high hs-CRP levels.

The exact mechanisms of the complex pathways of metabolic syndrome are not yet completely known. The pathophysiology is extremely complex and has been only partially elucidated. Most patients are older, obese, sedentary, and have a degree of insulin resistance. Stress can also be a contributing factor. The most important factors are weight, genetics, endocrine disorders such as polycystic ovary syndrome in women of reproductive age, aging and sedentary lifestyle, i.e., low physical activity and excess caloric intake. There is debate regarding whether obesity or insulin resistance is the cause of the metabolic syndrome or if they are consequences of a more far-reaching metabolic derangement. A number of markers of systemic inflammation, including C-reactive protein, are often increased, as are fibrinogen, interleukin 6 (IL-6), Tumor necrosis factor-alpha (TNF-α), and others. Some have pointed to a variety of causes including increased uric acid levels caused by dietary fructose. It is common for there to be a development of visceral fat, after which the adipocytes (fat cells) of the visceral fat increase plasma levels of TNF-α and alter levels of a number of other substances (e.g., adiponectin, resistin, PAI-1). TNF-α has been shown not only to cause the production of inflammatory cytokines but possibly to trigger cell signaling by interaction with a TNF-α receptor that may lead to insulin resistance. Chronic inflammation contributes to an increased risk of hypertension, artherosclerosis and diabetes.

In specific embodiments, the pharmaceutical composition of the invention is suitable for the treatment, amelioration, prophylaxis or delaying the onset of diabetes type II, diabetes type I or any diabetes-related condition. The World Health Organization recognizes three main forms of diabetes mellitus: Type 1, Type 2, and gestational diabetes (occurring during pregnancy), which have different causes and population distributions. While, ultimately, all forms are due to the beta cells of the pancreas being unable to produce sufficient insulin to prevent hyperglycemia, the causes are different. Type 1 diabetes is usually due to autoimmune destruction of the pancreatic beta cells. Type 2 diabetes is characterized by insulin resistance in target tissues, this causes a need for abnormally high amounts of insulin and diabetes develops when the beta cells cannot meet this demand. Gestational diabetes is similar to type 2 diabetes in that it involves insulin resistance, hormones in pregnancy may cause insulin resistance in women genetically predisposed to developing this condition.

Acute complication of diabetes (hypoglycemia, ketoacidosis or nonketotic hyperosmolar coma) may occur if the disease is not adequately controlled. Serious long-term complications include cardiovascular disease (doubled risk), chronic renal failure, retinal damage (which can lead to blindness), nerve damage (of several kinds), and microvascular damage, which may cause impotence and poor healing. Poor healing of wounds, particularly of the feet, can lead to gangrene, which may require amputation.

The inventors, aware of the risks associated with elevated blood sugar, also contemplate embodiments wherein the composition of the invention comprises at least one of the peptides comprising a sequence denoted as SEQ ID NO. 1 and 9, any fragments, derivatives or analogs thereof, or a nucleic acid sequence encoding the same, any combinations or mixtures thereof or any composition comprising the same. Such a composition, which enhances glucose metabolism and increases glucose tolerance may lead to lowering of serum glucose levels.

More specifically, the composition may be a pharmaceutical composition for the treatment, amelioration, prophylaxis or delaying the onset of a cardiac disorder. Cardiac disorder is a general term that may be used interchangeably with the terms heart disease and cardiovascular disease. Cardiac disorder refers to any type of disease that inhibits normal functioning of the heart. More specifically, disorders that comprise cardiac disorders herein include, but are not limited to coronary heart disease, cardiomyopathy, cardiovascular disease, ischemic heart disease, heart failure, hypertensive heart disease, inflammatory heart disease and valvular heart disease.

According to other embodiments, the pharmaceutical composition modulates the Th1/Th2, Th3 cell balance in a subject in need thereof.

In particular embodiments, the pharmaceutical composition of the invention is for the treatment, amelioration, prophylaxis or delaying the onset of an immune-related disorder. An “Immune-related disorder” is a condition that is associated with the immune system of a subject, either through activation or inhibition of the immune system, or that can be treated, prevented or diagnosed by targeting a certain component of the immune response in a subject, such as the adaptive or innate immune response.

Examples of immune-related disorders include, but are not limited to, Ulcerative Colitis, Crohn's Disease, Irritable Bowel Disease (IBD), Alopecia Areata, Lupus, Anlcylosing Spondylitis, Meniere's Disease, Antiphospholipid Syndrome, Mixed Connective Tissue Disease, Autoimmune Addison's Disease, Multiple Sclerosis, Autoimmune Hemolytic Anemia, Myasthenia Gravis, Autoimmune Hepatitis, Pemphigus Vulgaris, Behcet's Disease, Pernicious Anemia, Bullous Pemphigoid, Polyarthritis Nodosa, Cardiomyopathy, Polychondritis, Celiac Sprue-Dermatitis, Polyglandular Syndromes, Chronic Fatigue Syndrome (CFIDS), Polymyalgia Rheumatica, Chronic Inflammatory Demyelinating, Polymyositis and Dermatomyositis, Chronic Inflammatory Polyneuropathy, Primary Agammaglobulinemia, Churg-Strauss Syndrome, Primary Biliary Cirrhosis, Cicatricial Pemphigoid, Psoriasis, CREST Syndrome, Raynaud's Phenomenon, Cold Agglutinin Disease, Reiter's Syndrome, Rheumatic Fever, Discoid Lupus, Rheumatoid Arthritis, Essential Mixed, Cryoglobulinemia Sarcoidosis, Fibromyalgia, Scleroderma, Grave's Disease, Sjogren's Syndrome, Guillain-Barre, Stiff-Man Syndrome, Hashimoto's Thyroiditis, Takayasu Arteritis, Idiopathic Pulmonary Fibrosis, Temporal Arteritis/Giant Cell Arteritis, Idiopathic Thrombocytopenia Purpura (ITP), IgA Nephropathy, Uveitis, Insulin Dependent Diabetes (Type I), Vasculitis, Lichen Planus, and Vitiligo. The compositions and delivery systems described herein can be administered to a subject to treat or prevent disorders associated with an abnormal or unwanted immune response associated the above diseases.

It is appreciated that the pharmaceutical composition of the invention may further comprise at least one additional therapeutic agent.

According to some embodiments, the therapeutic agent may be an immunomodulatory agent or a metabolism-modulating agent. More specifically, the agent may be an anti-inflammatory agent or an anti-diabetic agent.

The term “metabolism-modulating agent” relates to the increase or decrease of activity or response in relation to a control or the normal or baseline level of activity or response under certain conditions, in the context of metabolic processes, such as glycolysis, gluconeogenesis, lipolysis, lipogenesis, oxidative phosphorylation, energy balance, metabolic signal transduction, etc. It can also encompass the maintaining of a level of activity or response under conditions that would normally increase or decrease the level of activity of the peptide or response.

As indicated above, since the peptides of the invention surprisingly exhibit metabolic and immune modulatory effects, the invention further provides pharmaceutical compositions comprising at least one isolated polypeptide comprising an amino acid sequence denoted by any one of SEQ. ID. NO. 1, SEQ. ID. NO. 5 or SEQ. ID. NO. 9, and any fragments, derivatives or analogs thereof, or a nucleic acid sequence encoding the same, and a pharmaceutically acceptable carrier, diluent or excipient.

The phrase “pharmaceutical composition”, as used herein refers to a preparation of one or more of the active ingredients described herein with other chemical components such as physiologically suitable carriers and excipients. The purpose of a pharmaceutical composition is to facilitate administration of a compound to an organism.

As used herein the term “active ingredient” refers to peptides of the present invention accountable for the intended biological effect. It will be appreciated that a polynucleotide encoding a peptide of the present invention may be administered directly into a subject (as is, or part of a pharmaceutical composition) where it is translated in the target cells i.e. by gene therapy. Accordingly, the phrase “active ingredient” also includes such polynucleotides.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

A carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidinones, methyl cellulose, pro-gelatinized starch, hydroxypropyl methyl cellulose, microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate in storage, while those containing too little might not disintegrate at a desired rate or under desired conditions.

Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolate, corn, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate or Sterotes, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL03 (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated. A glidant such as colloidal silicon dioxide can also be used.

Pharmaceutical compositions comprising the peptides of the present invention are useful for parenteral administration, i.e., intraperitoneally (i.p.), subcutaneously (s.c.), intramuscularly (i.m.) and intravenously (i.v.), as well as for oral and topical application. The compositions for parenteral administration commonly comprise a solution of the peptides or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate. The concentration of the peptides of the invention in these formulations can vary widely, i.e., from less than about 0.01%, usually at least about 0.1% to as much as 5% by weight and will be selected primarily based on fluid volumes, and viscosities in accordance with the particular mode of administration selected.

More specifically, injectable compositions that include the peptides of the invention may be prepared in water, saline, isotonic saline, phosphate-buffered saline, citrate-buffered saline, and the like and may optionally be mixed with a nontoxic surfactant. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is a sterile fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the desired particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. Isotonic agents such as sugars, buffers, or sodium chloride may be included. Prolonged absorption of the injectable compositions can be brought about by the inclusion in the composition of agents delaying absorption, for example, aluminum monosterate hydrogels and gelatin. Solubility enhancers may be added.

Sterile injectable compositions may be prepared by incorporating the peptides of the invention in the desired amount in the appropriate solvent with various other ingredients, e.g. as enumerated above, and followed by sterilization, as desired, by, for example filter sterilization. In the case of sterile powders for the preparation of sterile injectable solutions, methods of preparation include vacuum drying and freeze-drying techniques, which yield a powder of the active ingredient plus any additional desired ingredient present in a previously sterile-filtered solution. Any suitable sterilization process may be employees, such as filter sterilization, e.g. 0.22 micron filter or nanofiltration, gamma or electron beam sterilization.

In various embodiments, the final solution is adjusted to have a pH between about 4 and about 9, between about 5 and about 7, between about 5.5 and about 6.5, or about 6. The pH of the composition may be adjusted with a pharmacologically acceptable acid, base or buffer.

Still further, the compositions of the invention may be presented in unit dose forms containing a predetermined amount of each active ingredient per dose. Such a unit may be adapted to provide 0.1-100 mg/Kg of body weight of the peptides of the invention. Specifically, either 0.1-10 mg/Kg, 5-15 mg/Kg, 10-30 mg/Kg, 25-50 mg/Kg 40-80 mg/Kg or 60-100 mg/Kg. More specifically, said effective dosage is about 0.01 to about 100 mg/Kg of peptides, about 0.1 to about 90 mg/Kg, about 0.3 to about 8 mg/Kg, about 0.4 to about 70 mg/Kg, about 0.5 to about 60 mg/Kg, about 0.7 to about 50 mg/Kg, about 0.8 to about 40 mg/Kg, about 0.9 to about 30 mg/Kg, about 1 to about 20 mg/Kg, specifically, about 1 to about 10 mg/Kg. Such doses can be provided in a single dose or as a number of discrete doses. The ultimate dose will of course depend on the condition being treated, the route of administration and the age, weight and condition of the patient and will be at the doctor's discretion.

As indicated above, in addition to the parenteral route, the compositions of the invention may be adapted for administration by any other appropriate route, for example by the oral (including buccal or sublingual), rectal, nasal, topical (including buccal, sublingual or transdermal) or vaginal route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets, powders or granules, solutions or suspensions in aqueous or non-aqueous liquids, edible foams or whips, or oil-in-water liquid emulsions or water-in-oil liquid emulsions.

Pharmaceutical formulations adapted for transdermal administration may be presented as discrete patches intended to remain in intimate contact with the epidermis of the recipient for a prolonged period of time.

Pharmaceutical formulations adapted for topical administration may be formulated as ointments, creams, suspensions, lotions, powders, solutions, pastes, gels, sprays, aerosols or oils.

For applications to the eye or other external tissues, for example the mouth and skin, the formulations are preferably applied as a topical ointment or cream. When formulated in an ointment, the active ingredient may be employed with either paraffin or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for topical administration to the skin include ointment, cream, suspensions, paste, lotions, powders, solutions, oils, encapsulated gel, liposomes containing the peptides of the invention, any nano-particles containing the peptides, or sprayable aerosol or vapors containing said peptides. Conventional pharmaceutical carriers, aqueous, powder or oily bases, thickeners and the like may be necessary or desirable. The term “topically applied” or “topically administered” means that the ointment, cream, emollient, balm, lotion, solution, salve, unguent, or any other pharmaceutical form is applied to some or all of that portion of the skin of the patient skin that is, or has been, affected by, or shows, or has shown, one or more symptoms of the treated condition, for example, diabetes related skin injuries.

In certain embodiments, the administration of peptides of the invention for the treatment of skin disorders, specifically skin ulcers related to diabetes, may be by topical dressing. The term “dressing” means a covering for a wound or surgical site, typically composed of a cloth, fabric, synthetic membrane, gauze, or the like. It is usually a polymer-containing matrix covering an area of the skin. The dressing may or may not be in intimate contact with the skin. It can be, for example, a cloth or gauze, or it can be a polymer solution painted or sprayed on the skin, the polymer solidifying on the skin when the solvent dries off and/or when the polymer crosslinks. Dressings also include gels, typically cross-linked hydrogels, which are intended principally to cover and protect wounds, surgical sites, and the like.

Pharmaceutical formulations adapted for rectal administration may be presented as suppositories or enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists which may be generated by means of various types of metered dose pressurized aerosols, nebulizers or insufflators.

Pharmaceutical formulations adapted for vaginal administration may be presented as pessaries, tampons, creams, gels, pastes, foams or spray formulations.

Preferred unit dosage formulations are those containing a daily, biweekly, weekly, every few weeks or monthly dose or sub-dose, as herein above recited, or an appropriate fraction thereof, of an active ingredient.

It should be understood that in addition to the ingredients particularly mentioned above, the formulations may also include other agents conventional in the art having regard to the type of formulation in question, for example those suitable for oral administration may include flavoring agents.

In a further aspect, the invention provides a method for the treatment, amelioration, prophylaxis or delaying the onset of at least one of a metabolic disorder and an immune-related disorder. The method comprises the step of administering to a subject in need thereof a therapeutically effective amount of at least one isolated polypeptide comprising an amino acid sequence denoted by any one of SEQ. ID. NO. 1, SEQ. ID. NO. 5 or SEQ. ID. NO. 9, or any fragments, derivatives or analogs thereof, a nucleic acid sequence encoding the same, any combinations or mixtures thereof or any composition comprising the same.

The terms “treating” and “ameliorating” as used herein and in the claims mean improving one or more clinical indicia of disease activity in a patient having a pathologic disorder. “Treatment” refers to therapeutic treatment. Those in need of treatment are mammalian subjects suffering from metabolic or inflammatory disorders. By “patient” or “subject in need” is meant any mammal for which administration of the peptides of the invention, or any pharmaceutical composition of the invention is desired, in order to prevent, overcome or slow down such infliction. The peptides and compositions of the invention may also be administered to delay the onset of a disorder, that is, the peptides and compositions of the invention postpone the deterioration of the disorder or slow down its progress such that clinical signs of the disorder would appear later than they would without treatment.

To provide a “preventive treatment” or “prophylactic treatment” is acting in a protective manner, to defend against or prevent something, especially a condition or disease.

To achieve these therapeutic goals, a therapeutically-effective amount of the compositions or peptides of the invention must be administered to a subject suffering from said disorders. The terms “effective amount” or “sufficient amount” mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician).

As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

The present invention relates to the treatment of subjects, or patients, in need thereof. By “patient” or “subject in need” it is meant any organism who may be affected by the above-mentioned conditions, and to whom the treatment methods herein described are desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the methods and compositions of the invention are intended for mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. It should be further noted that particularly in case of human subject, administering of the compositions of the invention to the patient includes both self-administration and administration to the patient by another person.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician.

According to one embodiment of the method of the invention, the administered polypeptide comprises an amino acid sequence denoted by SEQ. ID. NO. 1, or any fragments, derivatives or analogs thereof.

According to another embodiment of the method of the invention, the administered polypeptide comprises an amino acid sequence denoted by SEQ. ID. NO. 5, or any fragments, derivatives or analogs thereof.

According to yet another embodiment of the method of the invention, the administered polypeptide comprises an amino acid sequence denoted by SEQ. ID. NO. 9, or any fragments, derivatives or analogs thereof.

In certain embodiments, the method of the invention leads to at least one of: enhancing glucose metabolism, increasing glucose tolerance, reducing plasma glucagon levels, inducing insulin receptor expression, increasing GLUT-4 glucose transporter expression, reducing serum levels of TNFα and reducing serum levels of IL-1β.

The net effect of the administered peptides is anti-inflammatory and anti-diabetic.

In fact, the method of the invention may be particularly suitable for the treatment, amelioration, prophylaxis or delaying the onset of metabolic syndrome or of any of the conditions comprising the same.

In certain embodiments, it should be noted that Metabolic Syndrome or any of the conditions comprising the same may be at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

More specifically, the method of the invention may be suitable for the treatment, amelioration, prophylaxis or delaying the onset of diabetes type II, diabetes type I or any diabetes related condition.

In one embodiment, the method of the invention comprises the administration of a therapeutically effective amount of at least one of the peptide comprising a sequence denoted as SEQ ID NO. 1 and 9, any fragments, derivatives or analogs thereof, or a nucleic acid sequence encoding the same, any combinations or mixtures thereof or any composition comprising the same. This method, which is effective in enhancing glucose metabolism and increasing glucose tolerance, may lead to the lowering of serum glucose levels.

It should be noted that these peptides, also referred to herein as APC2 and APC4, were shown by the present invention as expressed by a heart tissue. Moreover, the peptides of the invention were shown as down regulating glucose levels. Since of the main risk factors related to poor prognosis after heart attack is high glucose levels, the use of the peptides of the invention may enable protection after heart attack. Therefore, in more specific embodiments, the invention provides a method that may be suitable for the treatment, amelioration, prophylaxis or delaying the onset of a cardiac disorder, specifically, heart attack.

In some embodiments, the method modulates the Th1/Th2, Th3 cell balance in a subject in need thereof.

Accordingly, the method may be applicable for the treatment, amelioration, prophylaxis or delaying the onset of an immune-related disorder.

In certain specific embodiments, the peptides of the invention may modulate the Th1/Th2, Th3 cell balance towards an anti-inflammatory Th2 response. Th1/Th3 response may be particularly applicable in immune related disorders having an undesired unbalanced pro-inflammatory Th1 reaction. For example, such immune-related disorders may be an autoimmune disease, graft rejection pathology and an inflammatory disease.

In one embodiment, the method of the invention further comprises the step of administering at least one additional therapeutic agent.

The additional therapeutic agent may be any anti-inflammatory agent, including NSAIDs and steroidal agents, or any anti-diabetic agents.

In a further aspect, the invention is directed to the use of at least one isolated polypeptide comprising an amino acid sequence denoted by any one of SEQ. ID. NO. 1, SEQ. ID. NO. 5 or SEQ. ID. NO. 9, and any fragments, derivatives or analogs thereof, or a nucleic acid sequence encoding the same, in the preparation of a composition. The composition is suitable for the treatment, amelioration, prophylaxis or delaying the onset of at least one of a metabolic disorder and an immune-related disorder.

According to various embodiments, the polypeptide used in the preparation of the composition comprises an amino acid sequence denoted by SEQ. ID. NO. 1, or any fragments, derivatives or analogs thereof.

According to a number of embodiments, the polypeptide used in the preparation of the composition comprises an amino acid sequence denoted by SEQ. ID. NO. 5, or any fragments, derivatives or analogs thereof.

According to some embodiments, the polypeptide used in the preparation of the composition comprises an amino acid sequence denoted by SEQ. ID. NO. 9, or any fragments, derivatives or analogs thereof.

As demonstrated by the Examples, the composition prepared using the polypeptides of the invention may lead to at least one of: enhancing glucose metabolism, increasing glucose tolerance, reducing plasma glucagon levels, inducing insulin receptor expression, increasing GLUT-4 glucose transporter expression, reducing serum levels of TNFα and reducing serum levels of IL-1β.

According to particular embodiments, the composition prepared using the polypeptides of the invention is for the treatment, amelioration, prophylaxis or delaying the onset of the metabolic syndrome or of any of the conditions comprising the same.

In certain embodiments, it should be noted that Metabolic Syndrome or any of the conditions comprising the same may be at least one of dyslipoproteinemia (hypertriglyceridemia, hypercholesterolemia, low HDL-cholesterol), obesity, NIDDM (non-insulin dependent diabetes mellitus), IGT (impaired glucose tolerance), blood coagulability, blood fibrinolysis defects and hypertension.

In specific embodiments, the composition prepared using the polypeptides of the invention is for the treatment, amelioration, prophylaxis or delaying the onset of diabetes type II, diabetes type I or any diabetes related condition.

Thus, in numerous embodiments, a use of the invention is contemplated according to which at least one of the peptides used may comprise the amino acid sequence denoted as SEQ ID NO. 1 and 9 (peptide APC2 and APC4, respectively), any fragments, derivatives or analogs thereof, or a nucleic acid sequence encoding the same, any combinations or mixtures thereof or any composition comprising the same are used in the preparation of a composition. This composition, which enhances glucose metabolism and increases glucose tolerance, may lead to lowering of serum glucose levels. It should be noted that the invention further encompasses the use of functional fragments of any of the peptides of the invention. Non limiting examples of functional fragments may include the peptides of SEQ ID NO. 13, 14 and 15, that are functional fragments of the peptides of SEQ ID NO. 1, 5, and 9, respectively.

More specifically, the composition prepared according to the use of the invention may, according to some embodiments, be effective as a pharmaceutical composition for the treatment, amelioration, prophylaxis or delaying the onset of a cardiac disorder.

In other embodiments, the composition prepared using the polypeptides of the invention modulate the Th1/Th2, Th3 cell balance in a subject in need thereof.

According to one embodiment, the composition prepared using the polypeptides of the invention is for the treatment, amelioration, prophylaxis or delaying the onset of an immune-related disorder.

In certain specific embodiments, the peptides of the invention may modulate the Th1/Th2, Th3 cell balance towards an anti-inflammatory Th2, Th1/Th3 response may be particularly applicable in immune related disorders having an undesired unbalanced pro-inflammatory Th1 reaction. For example, such immune-related disorders may be an autoimmune disease, graft rejection pathology and an inflammatory disease.

According to various embodiments, the composition prepared using the polypeptides of the invention further comprise at least one additional therapeutic agent.

In yet another aspect, the invention provides an isolated polypeptide for use in the treatment, amelioration, prophylaxis or delaying the onset of at least one of an immune-related disorder and a metabolic disorder. The polypeptide comprises an amino acid sequence denoted by any one of SEQ. ID. NO. 1, SEQ. ID. NO. 5 or SEQ. ID. NO. 9, and any fragments, derivatives or analogs thereof.

The invention also provides a method for at least one of enhancing glucose metabolism, increasing glucose tolerance, reducing plasma glucagon levels, inducing insulin receptor expression, increasing GLUT-4 glucose transporter expression, reducing serum levels of TNFα and reducing serum levels of the method comprises the step of administering to a subject in need thereof a therapeutically effective amount of at least one isolated polypeptide comprising an amino acid sequence denoted by any one of SEQ. ID. NO. 1, SEQ. ID. NO. 5 or SEQ. ID. NO. 9, or any fragments, derivatives or analogs thereof, a nucleic acid sequence encoding the same, any combinations or mixtures thereof or any composition comprising the same.

In a specific embodiment, the method is for enhancing glucose metabolism.

In another embodiment, the method is for increasing glucose tolerance.

In yet another embodiment, the method is for increasing GLUT-4 glucose transporter expression.

In a yet another embodiment, the method is for reducing serum levels of TNFα.

In a still another embodiment, the method is for reducing serum levels of IL-1β.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Materials

D(−) Mannitol (Prod: 291484B, BDH laboratory supplies)

PCR apparatus (MyCycle, BioRad, CA, USA)

TNFα and IL-1β ELISA (BioPlex Pro-mouse diabetes kit cat#:171-F7001M and mouse Th1/Th2 kit cat#:171-F7001M, BioRad, CA, USA)

Glucometer (Accucheck, Roche]

RNA-Save (cat#:01-891-1B, Biological Industries, Israel).

RNeasy mini kit (cat#:74106, Qiagen) EZ-first strand cDNA synthesis kit for RT-PCR (cat#:20-800-50, Biological Industries, Israel)

GoTaq Green Master Mix (Cat# M7122, Promega)

PBS (Catalog No. 02-023-5A, Biological industries)

Maxisorp 96-wells plate (Catalog No. 442404, NUNC, F96 Maxisorp).

TW-20 (Catalog No, 0777-1L, Amresco)

BSA (Catalog No. 160069, MP biomedicals,)

Goat anti-rabbit HRP conjugate antibody (Catalog No. 7074, Cell signaling)

TMB (3,3′,5,5′-tetramethybenzidine, Horseradish peroxidase substrate, Catalog No. ES001-500ML, Millipore)

H₂SO₄ (Catalog No. 5552540, Frutarom)

Methods

General Methods of Molecular Biology

A number of methods of the molecular biology art are not detailed herein, as they are well known to the person of skill in the art. Such methods include PCR, expression of cDNAs, transfection of human cells, and the like. Textbooks describing such methods are, e.g., Sambrook et al, (1989) Molecular Cloning, A Laboratory Manual, Cold Spring Harbor Laboratory, ISBN: 0879693096; F. M. Ausubel (1988) Current Protocols in Molecular Biology, ISBN: 047150338X, John Wiley & Sons, Inc. Furthermore, a number of immunological techniques are not in each instance described herein in detail, like for example Western Blot, as they are well known to the person of skill in the art. See, e.g., Harlow and Lane (1988) Antibodies: a laboratory manual, Cold Spring Harbor Laboratory.

Animals and Treatment Regime

Twenty 7-weeks old C57Bl/6 male mice (per experiment) were purchased from Harlan Biotech (Jerusalem, Israel). All mice were maintained in the animal facility room at the company facility. The mice were given standard laboratory chow and water ad libitum and kept in a 12-hour light/dark cycle. All animal experiments were carried out in accordance with the guidelines of the Israeli authority for animal experiments for Care and Use of Laboratory Animals and with the authority specific approval.

ELISA General Protocol

Enzyme-linked Immunosorbent Assays (ELISAs) combine the specificity of antibodies with the sensitivity of simple enzyme assays, by using antibodies or antigens coupled to an easily-assayed enzyme. ELISAs can provide a useful measurement of antigen or antibody concentration. An ELISA is a five-step procedure: 1) coat the microtiter plate wells with antigen diluted in PBS incubate ON 4 C and wash; 2) block all unbound sites to prevent false positive results in BSA/FCS in PBS incubate 1 h and wash; 3) add antibody to the wells incubate 1 h and wash; 4) add anti-human IgG conjugated to an enzyme incubate 1 h and wash; 5) reaction of a substrate with the enzyme to produce a coloured product, thus indicating a positive reaction.

FACS Protocol

Cells were washed and suspension was adjusted to a concentration of 1−5×10⁶ cells/ml in ice cold PBS, 10% FCS, 1% sodium azide. 0.1-10 μg/ml of the primary labeled antibody were added. If necessary, dilutions were prepared in 3% BSA/PBS. The suspension was incubated for at least 30 min at room temperature or 4° C., then washed cells 3 times by centrifugation at 400 g for 5 minutes and re-suspension in 500 μl to 1 ml of ice cold PBS, 10% FCS, 1% sodium azide. Cells were analyzed using a flow-cytometer.

ELISA Procedure:

A calibration curve from 2000 pg/ml to 31 pg/ml was prepared by serial dilutions of a standard protein in PBS. The samples were thawed quickly in 37° C. bath. 70 μl duplicates of each blood sample (no dilution) and 70 μl triplicates of the standard samples were loaded on a Maxisorp 96-wells plate, and incubated at 4° C. overnight with shaking. The liquid was then removed and the plates washed 4 times using a multi-pipette with 300 μl 0.05% TW-20 in PBS. For blocking, 300 μl of the blocking buffer (5% BSA in PBS) were loaded in each well, and the plates incubated at room temperature for 1 hour with shaking. The liquid was then removed and the plates washed 4 times using a multi-pipette with 300 μl 0.05% TW-20 in PBS. For detection, the specific antibody was diluted 1:250 in diluent (0.05% TW-20, 0.1% BSA in PBS), and a 100 μl of detection antibody were loaded in each well and incubated at room temperature for 2 hours with shaking. The liquid was then removed and the plates washed 4 times using a multi-pipette with 300 μl 0.05% TW-20 in PBS. Goat anti-rabbit HRP conjugated antibody was diluted 1:200 in diluent, and a 1000 of HRP conjugate was loaded in each well and incubated for 30 minutes at room temperature with shaking. The liquid was then removed and the plates washed 5 times using a multi-pipette with 300 μl 0.05% TW-20 in PBS. For development, 1000 TMB (3,3′,5,5′-tetramethybenzidine, Horseradish peroxidase substrate) were added to each well and incubated until a blue color developed. To stop development, 50 μl 2N H₂SO₄ were added. Absorbance was determined at 450 nm in a microplate reader.

Glucose Tolerance Test

At the end of experiment (two weeks) a glucose tolerance test (GTT) was performed. The mice were starved overnight prior to administration of the test. Glucose was injected intraperitoneally (i.p.) (2 g/Kg body weight) and blood glucose levels were measured at time-points 0 min, 15 min, 30 min, 60 min, 90 min and 120 min. Blood glucose was determined by sampling tail vein blood using a standard glucometer.

Plasma Glucagon Assay

Mice tail vein was punctured and blood glucose was assayed using a standard glucometer.

Insulin Receptor and GLUT-4 PCR Analysis

Mice were sacrificed and dissected. 15-20 mg of appropriate tissue was taken and stabilized in RNA-Save. RNA was extracted from the tissue using an RNeasy mini kit. Homogenization was performed using a 22G needle and syringe. cDNA was prepared from the RNA using EZ-first strand cDNA synthesis kit for RT-PCR. PCR of samples was performed using GoTaq Green Master Mix using appropriate primers (40 cycles, annealing temperature: 62° C.).

Serum TNFα and IL-1β Assay

Blood was drawn from each mouse and collected separately in a 1.5 ml vial. After 30 min to 1 hr at room temperature, the clotted blood was centrifuged at 300G for 15 minutes at 4° C. The supernatant was collected to a new 1.5 ml vial and centrifuged again at 300G for 15 minutes. The supernatant was collected to a new 1.5 ml vial and frozen at −70° C. for later analysis. When ready for analysis, the blood vials were quickly thawed in a water bath at room temperature, and then centrifuged at 13,000 G for 10 minutes at 4° C. Samples were analyzed according to the Bio-Plex Pro assay instruction manual using a flat bottom black plate.

Reverse Transcriptase PCR (RT-PCR)

RT-PCR analysis was performed in various tissues, in order to establish the pattern of expression of the novel proteins isolated. The PCR conditions applied were 95° C. for 2 minutes, followed by 40 cycles of: 95° C. for 45 seconds, 59° C. for 45 seconds and 72° C. for 5 minutes, with an end cycle of 72° C. for 5 minutes.

### Example 1

### Isolation of APC-2 cDNA

A cDNA having the sequence denoted as SEQ ID NO.: 2, encoding the peptide APC-2 (having the sequence denoted as SEQ ID NO.: 1) was amplified using a human cDNA library purchased from Clonetech and the primers denoted here as SEQ ID NOs.: 3 and 4.

With respect to the APC-2 peptide, also named Uzi-1 herein, a maximal cleavage site probability of p=0.760 was determined between amino acid positions 17 and 18

The product of the PCR was sequenced. The PCR products were analyzed on agarose gels and stained with Cyber Green (Invitrogene), and the intensity of the PCR product was evaluated using BioRad ChemiDoc analyzer. The results demonstrated that APC-2 is expressed in the human heart and in lymphocytes.

### Example 2

### APC-2 Improves Glucose Tolerance

Male C57131/6 mice were divided into four groups of 5 mice each. The control group (Group I) received 5% Mannitol IV (200 μL 3 times a week). Group II received a dose of APC2 (0.25 mg/Kg body weight) 3 times a week. Group III received a dose of APC2 (1.5 mg/Kg body weight) 3 times a week. Group IV received a dose of APC2 (15 mg/Kg body weight) 3 times a week. The mice were treated for 2 weeks.

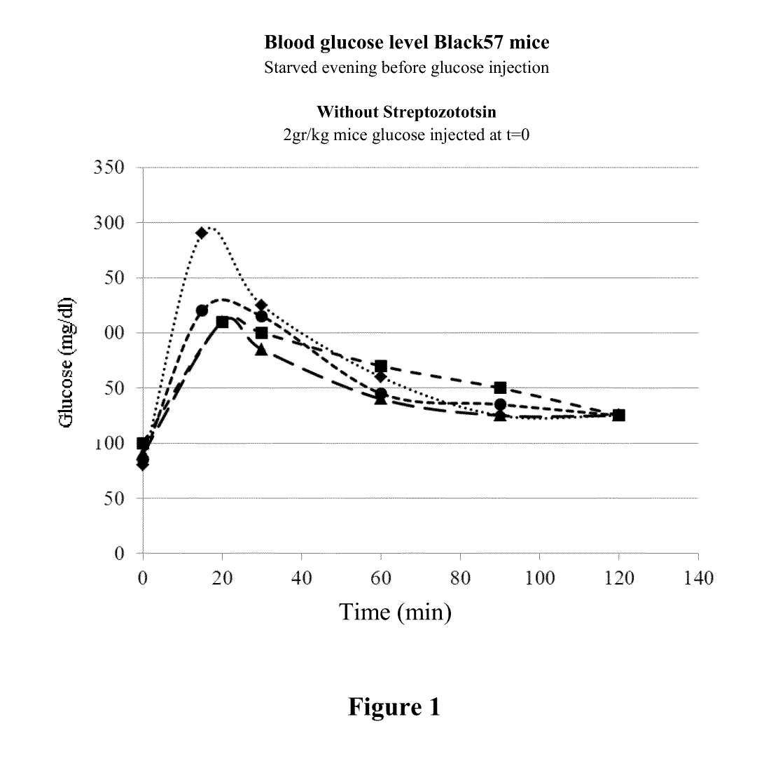

At the end of the experiment, a glucose tolerance test (GTT) was administered as described in the Methods section. The results of the GTT are presented in FIG. 1. As can be clearly seen, APC-2 improved glucose tolerance in a dose-dependent manner, with optimal response observed for 1.5 mg/Kg body weight APC2 dosage.

### Example 3

### APC-2 Reduces Plasma Glucagon

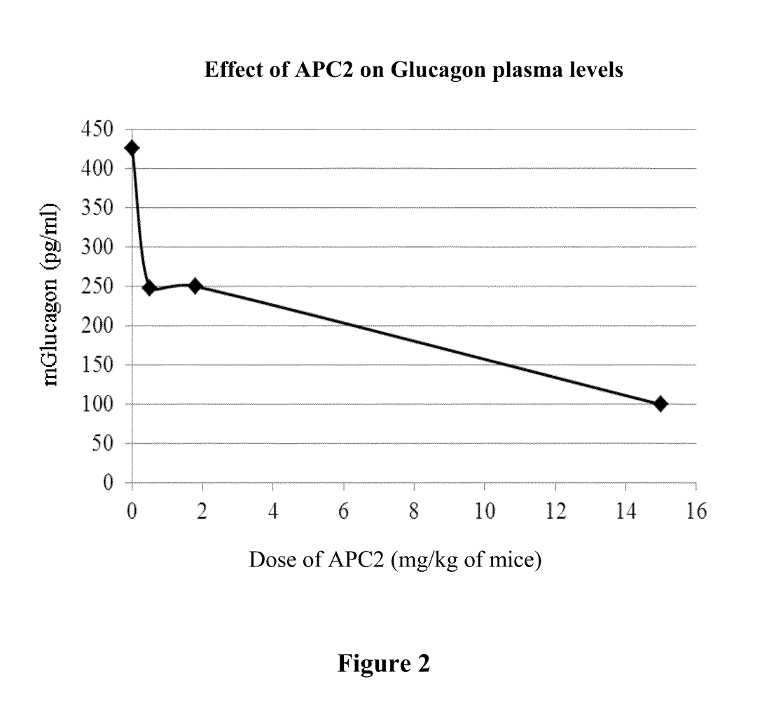

In addition to GTT, the mice plasma glucagon levels were assayed as described in the Methods section. Plasma glucagon levels, as shown in FIG. 2, were reduced in a dose-dependent manner by APC-2.

### Example 4

### APC-2 Increases Insulin Receptor and GLUT-4 Expression in Splenocytes

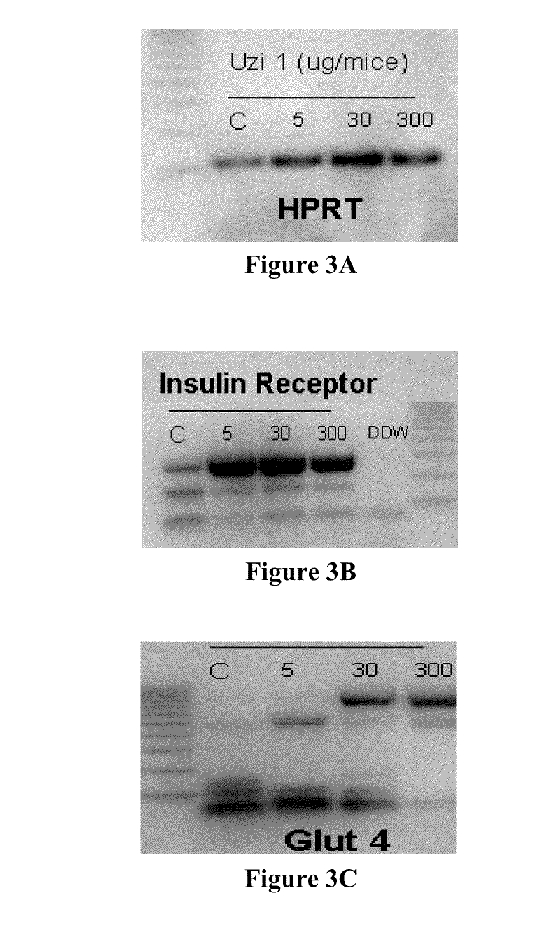

At the end of the experiment, the mice were sacrificed, the splenocytes were collected from the different mice groups and the RNA levels of HPRT (control), insulin receptor and GLUT4 were assayed. Results of these Northern blot analyses are presented in FIGS. 3A-3C. As illustrated, both insulin receptor and GLUT4 were up-regulated in mice treated with APC-2 in a dose-dependent manner, with maximal induction observed in mice treated with 1.5 mg/Kg body weight.

The results presented by Examples 1-4 clearly demonstrate the feasibility of using the APC-2 peptide of the invention for treating glucose metabolism associated disorders.

### Example 5

### Isolation of APC-3 cDNA

A cDNA having the sequence denoted as SEQ ID NO.: 6, encoding the peptide APC-3 (having the sequence denoted as SEQ ID NO.: 5) was amplified using a human cDNA library purchased from Clontech and the primers denoted here as SEQ ID NOs.: 7 and 8.

With respect to the APC-3 peptide, also named Uzi-2 herein, a maximal cleavage site probability of p=0.544 was determined between amino acid positions 18 and 19.

The product of the PCR was sequenced. The PCR products were analyzed on agarose gels and stained with Cyber Green (Invitrogen), and the intensity of the PCR product was evaluated using BioRad ChemiDoc analyzer. The results demonstrated that APC-3 is expressed in the human spleen, testis, small intestine, colon and kidney.

### Example 6

### APC-3 reduces TNFα and IL-1β

Male CS7Bl/6 mice were divided into four groups of 5 mice each. The control group (Group I) received 5% Mannitol IV (200 μL 3 times a week). Group II received a dose of APC3 (0.25 mg/Kg body weight) 3 times a week. Group III received a dose of APC3 (1.5 mg/Kg body weight) 3 times a week. Group IV received a dose of APC3 (15 mg/Kg body weight) 3 times a week. The mice were treated for 2 weeks.

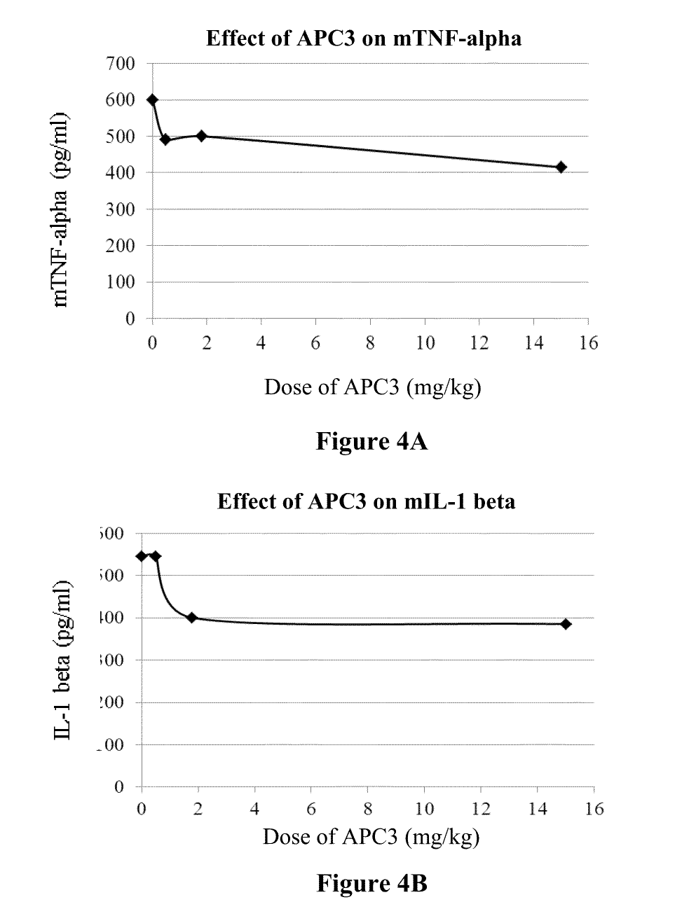

At the end of the experiment, TNFα and IL-1β were measured in the mice plasma, and the results are presented in FIGS. 4A and 4B, respectively. As clearly shown, APC-3 reduced both inflammatory cytokines levels in a dose-dependent manner, with optimal response observed for 1.5 mg/Kg body weight APC3 dosage.

It was therefore evident that APC3 can down-regulate pro-inflammatory cytokines (as manifested in the observed decrease in serum IL-1β and TNF-α), and therefore can be used for the treatment of autoimmune diseases and especially diabetes, since it is well known that high IL-1β is associated with diabetes.

### Example 7

### Isolation of APC-4 cDNA

A cDNA having the sequence denoted as SEQ ID NO.: 10, encoding the peptide APC-4 (having the sequence denoted as SEQ ID NO.: 9) was amplified using a human cDNA library purchased from Clontech and the primers denoted here as SEQ ID NOs.: 11 and 12.

With respect to the APC-4 peptide, also named Uzi-3 herein, a maximal cleavage site probability of p=0.731 was determined between amino acid positions 20 and 21.

The product of the PCR was sequenced. The PCR products were analyzed on agarose gels and stained with Cyber Green (Invitrogene), and the intensity of the PCR product was evaluated using BioRad ChemiDoc analyzer. The results demonstrated that APC-4 is expressed in the human spleen, testis, colon, small intestine, leukocytes, heart, placenta, liver, kidney and pancreas.

1-49. (canceled)

50. An isolated polypeptide comprising (a) the amino acid sequence set forth in SEQ ID NO: 1; (b) the amino acid sequence set forth in SEQ ID NO: 13; or (c) an amino acid sequence that is at least 80% identical to SEQ ID NO: 1 or SEQ ID NO: 13.

51. The isolated polypeptide of claim 50, consisting of the amino acid sequence set forth in SEQ ID NO: 1 or SEQ ID NO: 13.

52. An isolated nucleic acid molecule comprising a sequence encoding the polypeptide of claim 50.

53. The isolated nucleic acid molecule of claim 52, comprising the nucleic acid sequence set forth in SEQ ID. NO. 2.

54. An expression vector comprising the nucleic acid molecule of claim 52.

55. A host cell transformed or transfected with the expression vector of claim 54.

56. A pharmaceutical composition comprising an isolated polypeptide according to claim 50 and at least one pharmaceutically acceptable carrier, diluent or excipient.

57. A method for improving glucose metabolism in a subject suffering from a glucose metabolism related disorder, said method comprising administering an effective amount of an isolated polypeptide comprising (a) the amino acid sequence set forth in SEQ ID NO: 1; (b) the amino acid sequence set forth in SEQ ID NO: 13; or (c) an amino acid sequence that is at least 80% identical to SEQ ID NO: 1 or SEQ ID NO: 13; or a pharmaceutical composition comprising the same.

58. The method of claim 57, wherein said glucose metabolism related disorder is associated with hyperglycemia.

59. The method of claim 58, wherein said glucose metabolism related disorder associated with hyperglycemia is selected from the group consisting of diabetes mellitus type I, diabetes mellitus type II, gestational diabetes, atherosclerosis, diabetic retinopathy, metabolic syndrome, immune-related disorders and cardiac disorders.

60. The method of claim 57, wherein said method improves glucose tolerance.

61. The method of claim 57, wherein said method reduces plasma glucagon.

62. The method of claim 57, wherein said method induces the expression of insulin receptors.

63. The method of claim 57, wherein said method increases the expression of GLUT-4 glucose transporter.


SEQUENCE LISTING (15 sequences)

SEQ ID NO: 1 - protein, 29 aa, artificial
  Homo sapiens APC2
MITSWFSLLPGSQEATATPPFPVVAGRKH

SEQ ID NO: 2 - DNA, 87 bp, Artificial
  Homo sapiens APC2 DNA
atgataacctcatggttttctcttcttcctgggtctcaggaagcgacggctactccacccttcccagtggttgctggcagaaaacat

SEQ ID NO: 3 - DNA, 23 bp, artificial
  APC2 sense primer
atgataacctcatggttttctct

SEQ ID NO: 4 - DNA, 23 bp, artificial
  APC2 antisense primer
atgttttctgccagcaaccactg

SEQ ID NO: 5 - protein, 41 aa, Artificial Sequence
  Homo sapiens APC3
MSSFCLALEVFHPTVSSGRSRPYQIASLETPVLNYSFHHRR

SEQ ID NO: 6 - DNA, 123 bp, Artificial Sequence
  Homo sapiens APC3 DNA
atgtcctcattctgccttgctttagaggtattccaccccactgtctcttcagggagatcacgcccctaccagatagcctccctggagaccccagtcctcaactacagcttccatcacagaaga

SEQ ID NO: 7 - DNA, 24 bp, artificial
  APC3 sense primer
atgtcctcattctgccttgcttta

SEQ ID NO: 8 - DNA, 24 bp, artificial
  APC3 antisense primer
tcttctgtgatggaagctgtagtt

SEQ ID NO: 9 - protein, 47 aa, Artificial Sequence
  Homo sapiens APC4
MASMLSSFLLPVIYPEAASPTTPMSWNTCLGEVEGLKGYRAVLGREK

SEQ ID NO: 10 - DNA, 141 bp, Artificial Sequence
  Homo sapiens APC4 DNA
atggcctcaatgctgtcatcctttttgcttcctgttatctacccagaagcagcatccccaactacacctatgtcatggaacacttgtttaggtgaggtggaaggcctgaagggctacagggcagtgttagggagggaaaag

SEQ ID NO: 11 - DNA, 24 bp, Artificial Sequence
  APC4 sense primer
atggcctcaatgctgtcatccttt

SEQ ID NO: 12 - DNA, 24 bp, Artificial
  APC4 antisense primer
cttttccctccctaacactgccct

SEQ ID NO: 13 - protein, 14 aa, Artificial Sequence
  APC2 fragment
TATPPFPVVAGRKH

SEQ ID NO: 14 - protein, 14 aa, Artificial Sequence
  APC3 fragment
LETPVLNYSFHHRR

SEQ ID NO: 15 - protein, 14 aa, Artificial Sequence
  APC4 fragment
EGLKGYRAVLGREK